(19) United States
(12) Patent Application Publication
(10) Pub. No.: US 2016/0287300 A1
(43) Pub. Date: Oct. 6, 2016

(54) RETROGRADED HAMMERTOE COMPRESSION SCREW IMPLANT AND METHODS OF IMPLANTING THE SAME

(71) Applicant: WRIGHT MEDICAL TECHNOLOGY, INC., Memphis, TN (US)

(72) Inventors: Daniel F. MCCORMICK, Memphis, TN (US); David HARNESS, Eads, TN (US); Shannon CUMMINGS, Hernando, MS (US)

(21) Appl. No.: 14/418,771

(22) PCT Filed: Mar. 28, 2014
(86) PCT No.: PCT/US14/32105

§ 371 Date: Jan. 30, 2015

(51) Int. Cl. A61B 17/86; A61B 17/72; A61F 2/42; A61B 17/88

(52) U.S. Cl. CPC A61B 17/863; A61B 17/8886; A61B 17/72; A61B 17/7225; A61B 17/7291; A61F 2/4225

(57) ABSTRACT

A method and device to correct hammertoes. The device is a bone implant that includes an elongated body having a first threaded portion with a first thread pitch and a second threaded portion with a second thread pitch. When the implant is implanted into a joint and rotated about its longitudinal axis when mated with a driver bit, a target joint may be compressed using the pitch differential between the first and second threads. The first and second threads may also be disposed at opposing angles, to help prevent pistoning of the compressed bones. The implant can have one or two driving heads, to accommodate the different methods of insertion. The method involves surgically opening the PIP joint, driving a first portion of a bone implant in one direction into one of the proximal and middle phalanges, aligning the toe such that the second portion of the implant is aligned with the other of the proximal and middle phalanges, and driving the implant in the opposite direction, using a driver bit inserted through the tip of the toe and through an intramedullary canal in the distal and middle phalanges, such that the second portion is driven into the other of the proximal and middle phalanges.

### FIELD OF DISCLOSURE

The disclosed device and method generally relate to hammertoe correction devices and methods of implanting those devices into a patient's toe.

### BACKGROUND

A hammertoe or contracted toe is a deformity of the proximal inter-phalangeal joint of the second, third, or fourth toe causing it to be permanently bent and giving it a semblance of a hammer. Initially, hammertoes are flexible and may be corrected with simple measures but, if left untreated, hammertoes may require surgical intervention for correction. Persons with hammertoe may also have corns or calluses on the top of the middle joint of the toe or on the tip of the toe and may feel pain in their toes or feet while having difficulty finding comfortable shoes.

Various treatment strategies are available for correcting hammertoes. Conventionally, the first line of treatment for hammertoes includes employing new shoes having soft and spacious toe boxes. Additionally, toe exercises may be prescribed to stretch and strengthen respective muscles, e.g., gently stretching one's toes manually, using the toes to pick up things off the floor, etc. Another line of treatment may include employing straps, cushions or non-medicated corn pads to relieve symptoms.

An addition method of treatment may include correction by surgery if other non-invasive treatment options fail. Conventional surgery usually involves inserting screws, wires or other similar implants in toes to straighten them. Traditional surgical methods generally include the use of Kirschner wires (K-wires). Due to various disadvantages of using K-wires, however, compression screws are being employed as a better implant alternative as K-wires require pings protruding through the end of respective toes due to their temporary nature. As a result, K-wires often lead to pin tract infections, loss of fixation, and other conditions. Additional disadvantages of K-wires include migration and breakage of the K-wires thus resulting in multiple surgeries.

Screw implants may provide a more permanent solution than K-wires as such implants do not need removal and have no protruding ends. Further, with the use of screw implants, a patient may wear normal footwear shortly after the respective surgery. There are generally two types of known screw implants: single-unit implants, which possess a completely threaded body and do not provide a flexibility to the respective toe in its movement, and articulated or two-unit implants, which typically have one unit that is anchored into the proximal phalanx, a second unit that is anchored into the distal phalanx, and a fitting by which the two units are coupled. Either or both of the two units may be threaded or have other anchoring structures such as barbs or splaying arms.

Among other disadvantages, both kinds of known implants result in an undesirable pistoning effect, i.e., part or all of the implant will toggle or move within the bone as the patient's toe moves. Pistoning decreases the stability of the implant and lessens the compression across the joint. Moving parts, such as fittings, hinges, expansion pieces, and the like also decrease the stability, lifespan, and compression force of the implant. Accordingly, there remains a need for durable hammertoe implants which are not only stable but provide adequate compression across a joint with minimal pistoning. There also remains a need for an implant which can provide these advantages, while being easily inserted with minimal damage to the surrounding tissue.

### BRIEF DESCRIPTION OF THE DRAWINGS

Other objects, features, and advantages of the present invention will be apparent from the following description when read with reference to the accompanying drawings. In the drawings, wherein like reference numerals denote corresponding parts throughout the several views.

FIG. 1 is a side view of an exemplary implant according to some embodiments of the present subject matter.

FIG. 2 is an illustration of an exemplary driver bit for use with some embodiments of the present subject matter.

FIG. 3 is a cutaway view of the driver bit shown in FIG. 2 loaded in a driver in one possible loading configuration.

FIG. 4 is a partial side exploded view of the male driving head shown in FIG. 2 engaging the implant shown in FIG. 1.

FIG. 5A is cutaway side view of the implant shown in FIG. 1 anchored in the proximal phalanx according to some embodiments of the present subject matter. FIG. 5B is a cutaway side view of the drilling spade shown in FIG. 2 preparing the middle phalanx for receiving the implant shown in FIG. 1. FIG. 5C is a cutaway side view of the driving male driving head shown in FIG. 2 protruding from the middle phalanx according to some embodiments of the present subject matter. FIG. 5D is a cutaway side view of the implant shown in FIG. 1 being anchored into the middle phalanx according to some embodiments of the present invention.

FIG. 6A is a side view of a another exemplary implant for use with some other embodiments of the present subject matter. FIG. 6B is a cutaway side view of the implant shown in FIG. 6A.

FIG. 7 is a side view of still another exemplary implant for use with still other embodiments of the present subject matter.

### SUMMARY

The present subject matter relates to a type of bone implant useful in the correction of hammertoe and similar maladies, as well as methods of inserting the implant into bones to effectuate that correction. The bone implant has a number of different embodiments, each of which correspond to different nuances in their respective methods of insertion. All of the implant embodiments have an elongated body with a first portion and a second portion. The first portion and second portion can represent the proximal and distal portions of the implant, or vice versa, depending on the desired orientation of the implant in the toe or other body part. The implant has attributes of a compression screw, in that generally, the first portion bears a first thread and the second portion bears a second thread. There may also be an unthreaded transition portion in between the first and second threaded portions.

In preferred embodiments, the first and second threads have different pitches such that one portion of the implant will travel a different distance than the other portion for each rotation of the implant. For example, the first thread pitch can be 0.039 inches and the second thread pitch can be 0.069 inches. For clarity and simplicity, the embodiments shown and described herein have first and second threads wound in the same direction. One skilled in the art will appreciate how to modify the methods of insertion to accommodate an implant that has first and second threads wound in opposite directions, which may be used in addition to or in lieu of thread pitch differential in order to create a compressive force.

Additionally, the first and second threads may be disposed at an angle or incline with respect to the axis of rotation of the body. In preferred embodiments, these angles are in an opposing configuration, such that the first and second threads are tilted towards each other, or towards the center of the body. An example of suitable angles for these embodiments is 25 degrees from vertical or perpendicular in each direction, or in other words, 65 degrees and 115 degrees from the longitudinal axis of the body. Configuring the threads at opposing angles helps reduce pistoning of the implant, and resists movement of the surrounding tissue against the compressive force created by the thread pitch differential.

In some embodiments, the first portion of the implant has a driving end adapted to mate with a driver bit. For instance, the driving end could define a female depression configured to mate with a male-headed driver bit. These embodiments are useful for insertion methods where the second portion is first driven into the proximal phalanx, and the first portion is then retrograde driven into the middle phalanx. Both driving actions involve mating the same driver bit with the single driving end. The second portion is driven into the proximal phalanx while the toe is bent and the PIP joint is surgically exposed, and then the first portion is driven in the opposite direction after the toe is straightened and the PIP joint is closed with the first portion aligned with the middle phalanx. When the toe is in this straight and closed configuration, the driver bit accesses the driving head through the tip of the toe, by way of an intramedullary canal drilled through the distal and middle phalanges. The thread pitch differential between the first and second threads creates a compressive force across the patient's proximal inter-phalangeal joint.

In other embodiments, the first portion of the elongated body bears a first thread and defines a first driving end. The second portion bears a second thread and defines a second driving end. The first and second threads may have different pitches and may also be disposed at opposing angles. The first driving end is adapted to mate with a first driver bit and the second driving end is adapted to mate with a second driver bit. The two driving ends can be identical and mate with the same driver bit, or one end can have a male extension and the other can have a female depression, to mate with corresponding driver bits. These embodiment are useful for insertion methods where the first portion is first driven into the middle phalanx, and the second portion is then driven into the proximal phalanx. In this method, an intramedullary canal is drilled through the distal and middle phalanges. A driver bit can then be inserted through the canal and mated to the first driving end. The first portion of the implant is then driven into the middle phalanx while the toe is bent and the PIP joint is surgically exposed, by mating the second driving end with a corresponding second driver bit and rotating the implant. The toe is then straightened and the PIP joint is closed with the second portion aligned with the proximal phalanx. The driver bit placed in the intramedullary canal and mated with the first driving end inside the middle phalanx is then rotated (in the opposite direction) to drive the second portion into the proximal phalanx. The thread pitch differential creates a compressive force across the patient's proximal inter-phalangeal joint.

The present subject matter also relates to a method of correcting hammertoes. Although the steps of the various embodiments of this method are described as being performed in a particular order, this is merely for clarity and simplicity, and one skilled in the art will appreciate that some steps may be reordered for convenience or preference. The first step of the method is to make a dorsal incision in a patient's toe along the patient's PIP joint, bending the patient's toe such that the PIP joint is open and a part of the proximal and middle phalanges are exposed. If desired, the proximal and middle phalanges may be prepared by resecting the bones or drilling intramedullary canals to serve as pilot holes for the portions of the implant that will be threaded into the bone. The next steps depend on which embodiment of the implant is being used.

For the particular embodiment with a single driving head, described above, insert the implant into the proximal phalanx, such that the first portion penetrates the proximal phalanx and the first thread engages bone tissue of the proximal phalanx. Drill an intramedullary canal completely through the distal and middle phalanges, from the tip of the toe to the PIP joint. Insert a driver bit adapted to mate with the driving end into the intramedullary canal through the tip of the toe. Straighten the patient's toe such that the second portion of the implant aligns with the intramedullary canal. Drive the bone implant into the middle phalanx by mating the driver bit with the driving end and rotating the implant in the opposite direction, until the second portion is anchored in the middle phalanx and the proximal inter-phalangeal joint is compressed. Withdraw the driver bit through the tip of the toe.

For the embodiment with two driving heads, described above, drill an intramedullary canal completely through the distal and middle phalanges, and insert a first driver bit into the intramedullary canal through the tip of the toe. Insert the first portion of the implant into the middle phalanx such that the first driving end penetrates the middle phalanx and mates with the driver bit, and the first thread engages bone tissue of the middle phalanx. Drive the first portion into the middle phalanx by mating a second driving bit with the second driving end and rotating the implant. Remove the second driver bit, but leave the first driver bit in place. Straighten the toe and align the implant with the second portion against the proximal phalanx. Drive the second portion of the implant into the proximal phalanx by rotating the first driver bit in the opposite direction until the PIP joint is compressed. Withdraw the driver bit through the tip of the toe.

### DETAILED DESCRIPTION

With reference to the figures, where like elements have been given like numerical designations to facilitate an understanding of the present subject matter, the various embodiments of a retrograded hammertoe compression screw implant are described.

It should be noted that the figures are not necessarily to scale and certain features may be shown exaggerated in scale or in somewhat schematic form in the interest of clarity and conciseness. In the description, relative terms such as “distal, “proximal,” “first,” “second,” “straight,” “bent,” “open,” “closed,” etc.) should be construed to refer to the orientation or designation as then described or as shown in the drawing figure under discussion. Terms including “into” “out,” “longitudinal,” “opposing,” and the like are to be interpreted relative to one another or relative to an axis of elongation, or an axis or center of rotation, as appropriate. Terms concerning attachments, coupling and the like, such as “connected” refer to a relationship wherein structures are secured or attached to one another either directly or indirectly through intervening structures, as well as both movable or rigid attachments or relationships, unless expressly described otherwise. The terms “implant” and “device” are used interchangeably in this disclosure and such use should not limit the scope of the claims appended herewith.

Embodiments of the present subject matter provide stability and compression across proximal or distal inter-phalangeal joints while maintaining the simplicity of a hammertoe fusion. Exemplary embodiments may feature a double-ended threaded device, each end having a thread pitch (disparate or otherwise) that, when implanted, provides compression across a targeted joint. Such embodiments may have one driving end or two, and may be solid or cannulated, depending on a surgeon's preferred method of insertion. Exemplary embodiments of the present subject matter may also feature methods of inserting double-ended threaded devices. Such embodiments may involve first driving the device into the proximal phalanx and then retrograde driving it into the middle phalanx, or vice versa, depending on the configuration of the device.

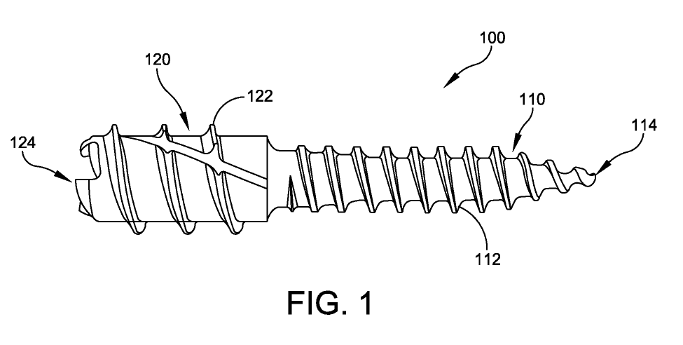

FIG. 1 is a side view of an exemplary implant according to some embodiments of the present subject matter. With reference to FIG. 1, an implant 100 for correcting hammertoes may comprise a proximal portion 110 and a distal portion 120. The proximal portion 110 includes proximal threads 112 on an external surface thereof having a first pitch, and the distal portion 120 includes distal threads 122 on an external surface thereof having a second pitch. In one embodiment, the proximal threads 112 on the proximal portion 110 have a pitch of 0.039 inches, and the distal threads 122 on the distal portion 120 have a pitch of 0.069 inches. Of course, these pitches are exemplary only and should not limit the scope of the claims appended herewith as the first and second thread pitches may be the same as each other and may be greater or lesser than the examples provided. The thread pitches may be threaded in substantially the same direction or in opposing directions and may or may not have different pitches. The implant 100 may be constructed of any suitable material such as stainless steel, titanium, or other metals or rigid polymers.

The proximal threads 112 are disposed on the external surface of the proximal portion 120 at a first angle or incline with respect to the implant's 100 longitudinal axis of rotation. Likewise, the distal threads 122 are disposed on the external surface of the distal portion 120 at a second angle or incline with respect to the implant's 100 axis of rotation. The first angle (i.e. the angle of the proximal threads 112) is inverted with respect to the second angle (i.e. the angle of the distal threads 122) such that the proximal and distal threads 112 and 122 are tilted towards each other. Such a thread angle configuration can be referred to as “opposing,” or reverse incline angles.

In one embodiment, the distal portion 120 may include a distal driving end 124 having a female depression adaptable to mate with a driver bit 200 (not shown in FIG. 1) having a male interface 210 (not shown in FIG. 1). The distal end 120 may instead include a male interface (not shown in FIG. 1) to mate with a driver bit 200 having a corresponding female depression. For example, the distal end 120 may have a portion in the shape of a hex whereby a suitable driver has a corresponding hex adapter appropriate to drive the implant 100 into a respective bone.

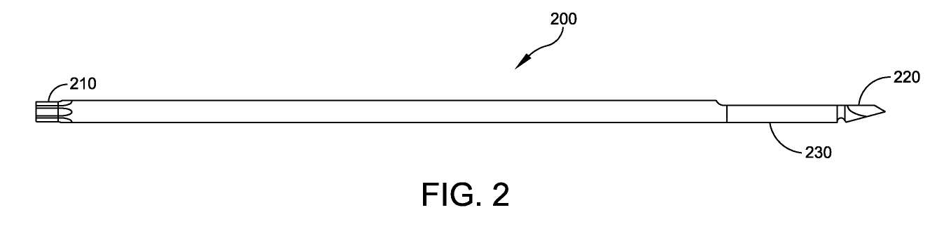

FIG. 2 is an illustration of an exemplary driver bit for use with some embodiments of the present subject matter. With reference to FIG. 2, an exemplary driver bit 200 may be an elongated instrument and include one end having an interface 210 suitable for mating with an implant 100 described above. In the illustrated example, the interface 210 comprises a male hexagonal head adaptable to mate to a corresponding female depression 124 in an implant 100. In one embodiment of the present subject matter, the male hexagonal head is a 2.0 mm hexagonal head. Of course, other geometries and interfacing mechanisms are envisioned and the male hexagonal head of the driver bit 200 and its noted dimensions should not limit the scope of the claims appended herewith. On an opposing end of the driver 200 may be a drilling spade 220 or trocar and may include a flat modular section 230 adaptable to accept a handle or other suitable mechanism to assist a surgeon during installation of an exemplary implant 100.

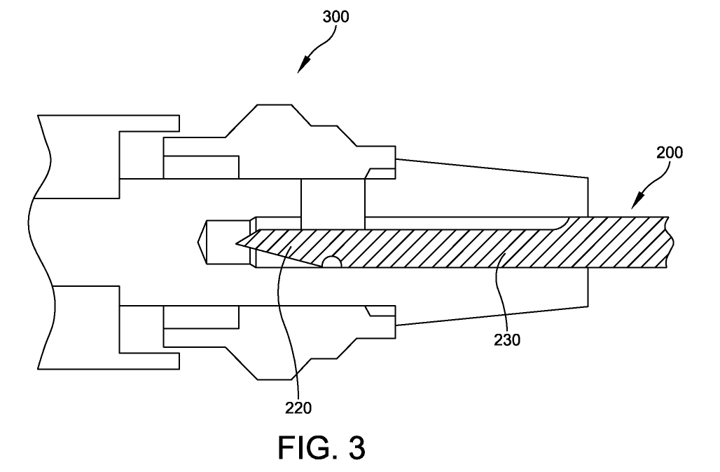

FIG. 3 is a cutaway view of the driver bit 200 shown in FIG. 2 loaded in a driver 300 in one possible configuration. With reference to FIG. 3, the drilling spade 220 end is housed within the driver 300, such that the interface 210 (not shown in FIG. 3) is available for mating with the distal driving end 124 (not shown in FIG. 3).

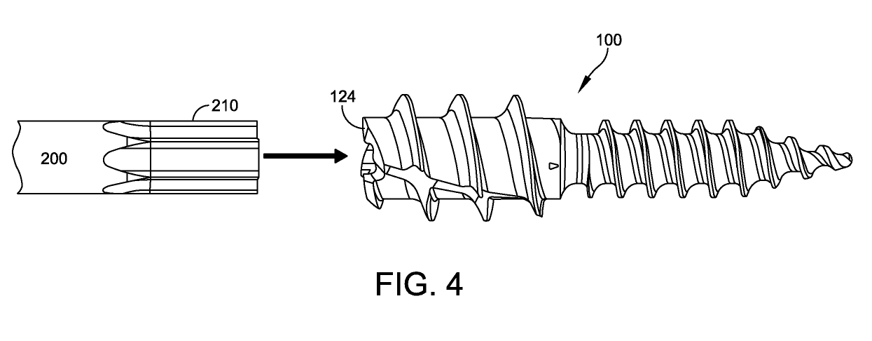

FIG. 4 is a partial side exploded view of the male interface 210 and the distal driving end 124 that are implied by but not shown in FIG. 3.

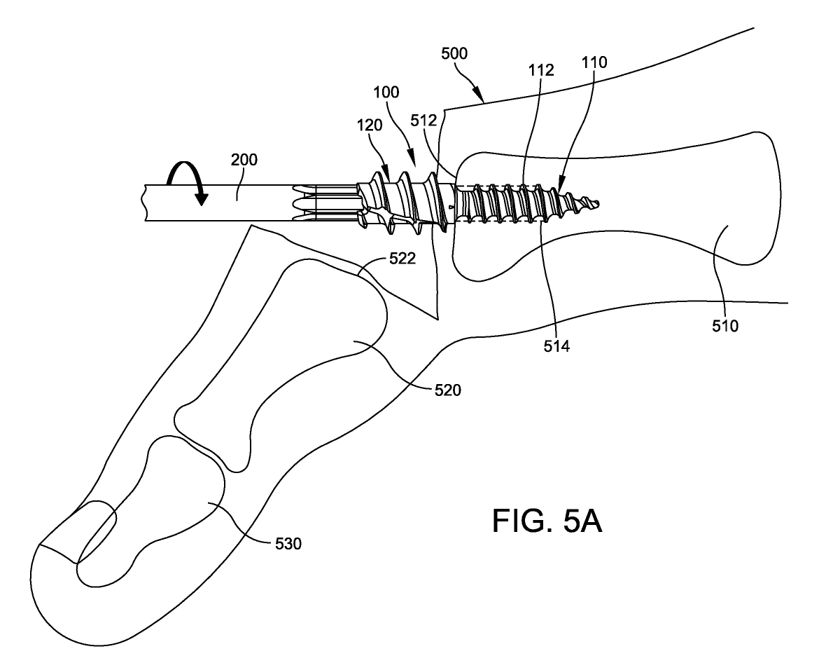

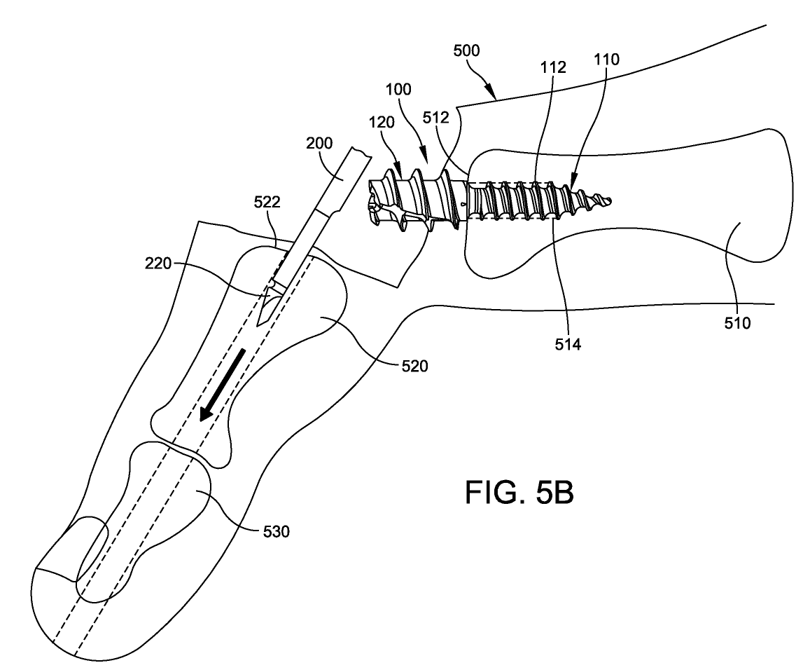

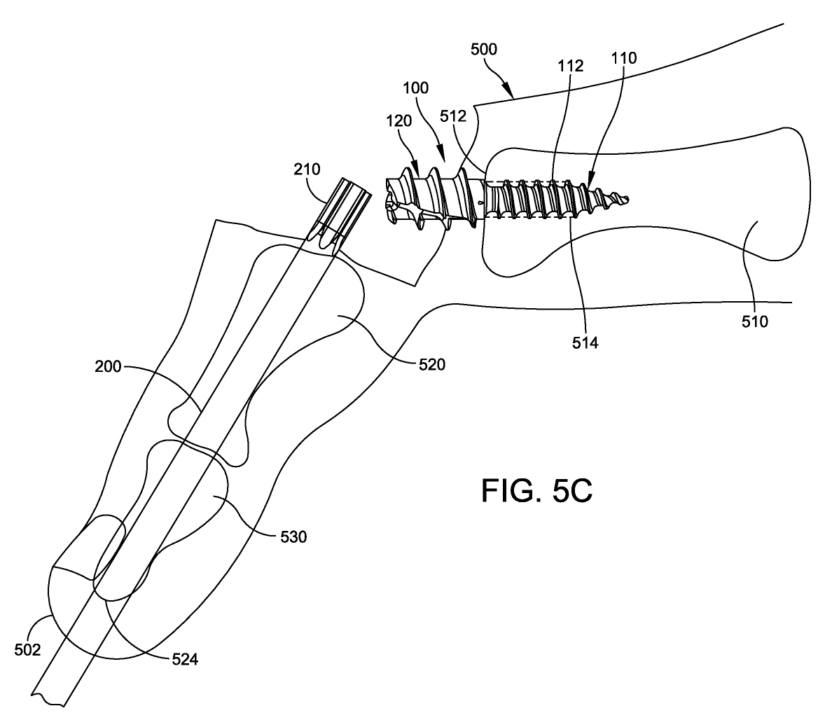

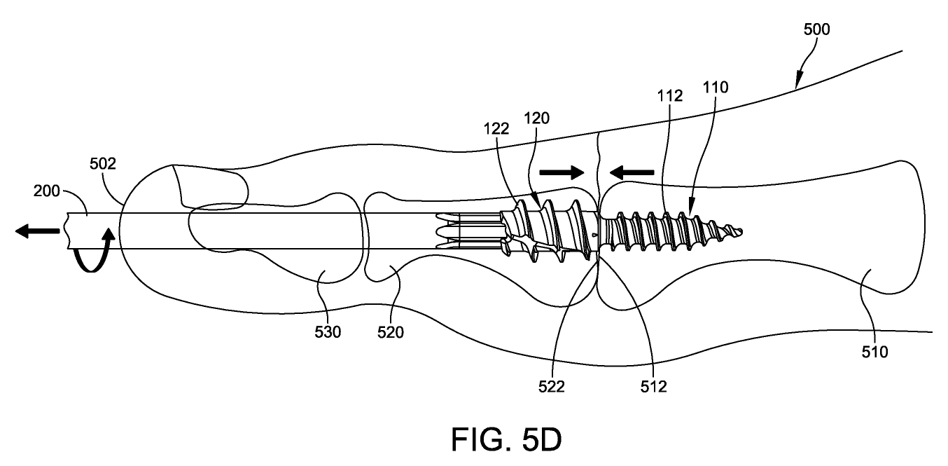

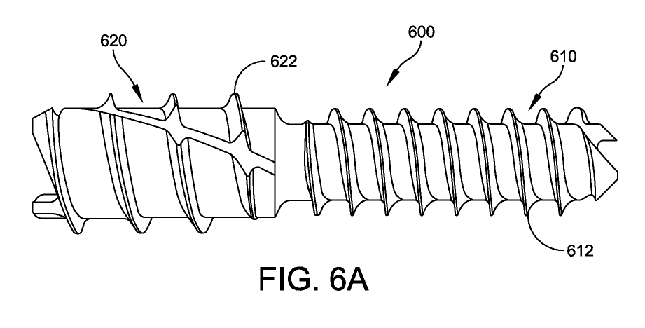

FIGS. 5A-5D illustrate an exemplary method of installation or implantation of an implant 100 according to some embodiments of the present subject matter. With reference to FIG. 5A, in one embodiment to install an implant 100, a toe 500 may be surgically opened to provide access to a proximal inter-phalangeal (PIP) joint between a proximal phalanx 510 and a middle phalanx 520. The proximal and middle bone surfaces 512 and 522, respectively, of the proximal 510 and middle 520 phalanges, may be resected using a bone saw or other tool if necessary. A proximal intramedullary canal 514 may be drilled into the proximal phalanx, if desired to prepare the proximal phalanx 510 for receiving the implant 100, using the drilling spade 220 (not shown in FIG. 5A) end of the driver bit 200 or another appropriate tool. The proximal intramedullary canal 514 should small enough that the proximal thread 110 cannot pass therethrough without engaging bone tissue of the proximal phalanx 510. The proximal portion 110 of the implant 100 is then driven, i.e. rotated about its longitudinal axis, into the proximal phalanx 510 such that the threads 112 penetrate the bone tissue of the proximal phalanx 510, until the distal portion 120 is just proud of the proximal bone surface 512.

With reference to FIG. 5B, the drilling spade 220 end of the driver bit 200 is then used to drill a distal intramedullary canal 524 into the middle bone surface 522 through the middle phalanx 520 and distal phalanx 530, and out the tip 502 of the toe 500. The distal intramedullary canal 524 should be large enough that the driver bit 200 can pass therethrough, but small enough that the distal thread 122 cannot pass therethrough without engaging bone tissue of the middle phalanx 520. In the exemplary embodiment shown in FIG. 5B, the proximal portion 110 of the implant 100 is already implanted in the proximal phalanx 510 at this time, but other embodiments in which the intramedullary canals 514 and 524 are pre-drilled are also contemplated by this subject matter.

With reference to FIG. 5C, the driver 300 (not shown in FIG. 5C) may be reconfigured as shown in FIG. 3, and the driver bit 200 inserted into the tip 502 of the toe 500, through the distal intramedullary canal 524, until the male interface 210 protrudes from the middle bone surface 522. The male interface 210 is then mated with the distal driving end 124 and the middle phalanx introduced to the distal portion 120 of the implant 100. The toe 500 is accordingly re-aligned in preparation for the middle phalanx 520 to receive the distal portion 120 of the implant 100.

With reference to FIG. 5D, the driver 300 (not shown in FIG. 5D) operates to rotate the driver bit 200 such that the distal portion 120 of the implant 100 is driven through the middle phalanx 520 in a retrograde fashion, and the distal threads 122 penetrate the bone tissue of the middle phalanx 520. Although the retrograde motion of the implant 100 being driven into the middle phalanx 520 may result in the proximal portion 110 partially backing out of the proximal phalanx 510, the thread pitch differential between the proximal and distal threads, 112 and 122, ensures that the proximal portion 110 retreats less with each turn than the distal portion 120 advances. The overall motion of the implant 100 thus pulls the middle phalanx 520 and proximal phalanx 510 towards each other until the proximal and middle bone surfaces, 512 and 522, are adjacent and the PIP joint is compressed. The implant 100 is optimally placed when the transition between the proximal portion 110 and distal portion 120 is aligned with the fused PIP joint. The driving bit 200 is then separated from the implant 100 and withdrawn through the tip 502 of the toe 500.

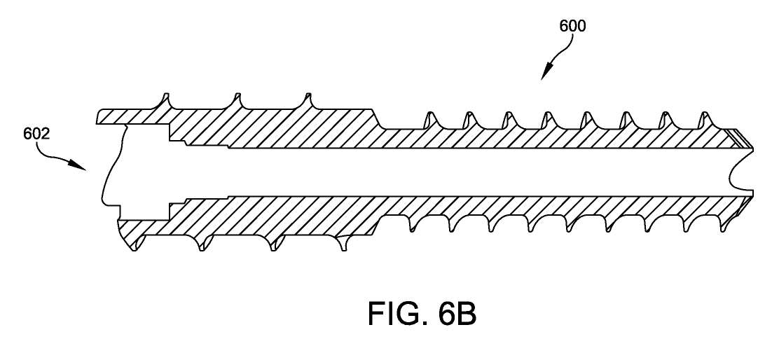

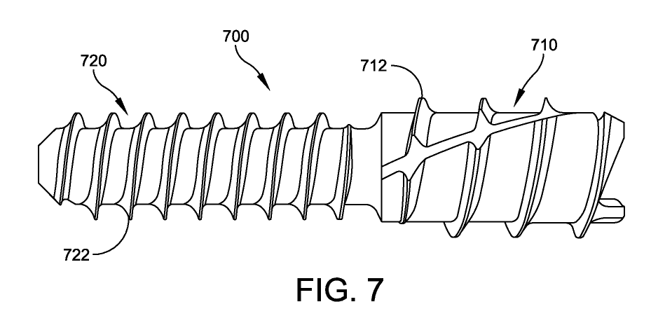

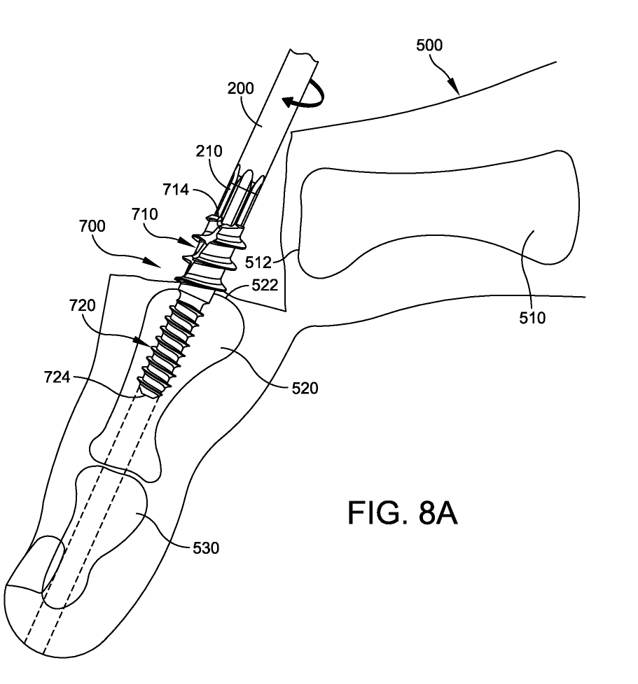

FIG. 6A is a side view of another exemplary implant for use with some other embodiments of the present subject matter. FIG. 6B is a cutaway side view of the implant shown in FIG. 6A. With reference to FIGS. 6A and 6B, a cannulated implant 600 defines a cannula 602 running along its longitudinal axis of rotation. The cannulated implant 600 is essentially a cannulated version of the implant 100 shown in FIGS. 1-5D. The cannulated implant 600 has a proximal portion 610 that includes proximal threads 612 on an external surface thereof having a first pitch and a first angle, and the distal portion 620 includes distal threads 622 on an external surface thereof having a second pitch and a second angle. The pitch differential, angle, and direction of the threads 612 and 622 of the cannulated implant 600 may be similar to that of the implant 100 shown in FIGS. 1-5D. The cannulated implant 600 may be constructed of any suitable material such as stainless steel, titanium, or other metals or rigid polymers. The method of insertion of the cannulated implant 600 is similar to the method of insertion of the implant 100 shown in FIGS. 5A-5D, with one exception being that the cannulated implant 600 is adapted for use with a K-wire or other appropriate guide wire. One of skill in the art will readily appreciate how to incorporate the use of a K-wire into the method described with reference to FIGS. 5A-5D, in the event that having the guidance of a K-wire is desired by the surgeon. Once the cannulated implant 600 is anchored into the proximal phalanx 510 and middle phalanx 520 in a position analogous to that shown of the implant 100 in FIG. 5D, the K-wire is removed through the tip 502 of the toe 500.

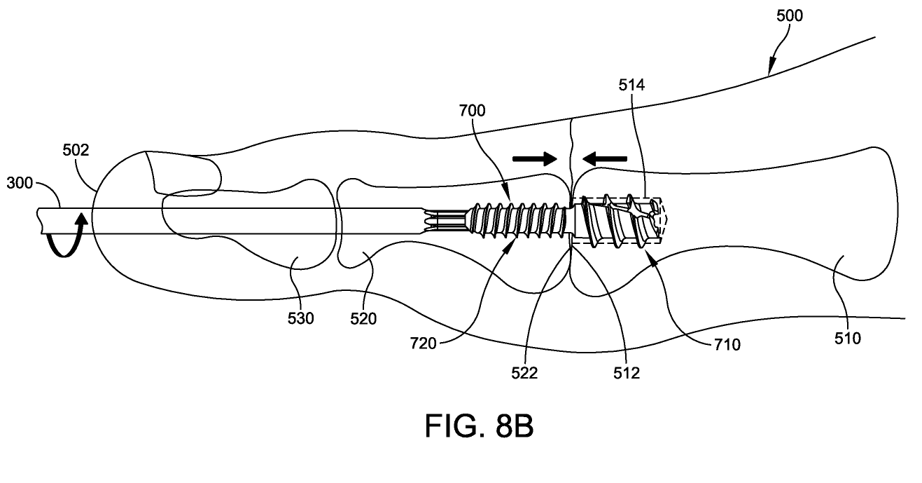

FIG. 7 is a side view of still another exemplary implant for use with still other embodiments of the present subject matter. With reference to FIG. 7, a dual-headed implant 700 for correcting hammertoes may comprise a proximal portion 710 and a distal portion 720. The proximal portion 710 includes proximal threads 712 on an external surface thereof having a first pitch and a first incline angle, and the distal portion 720 includes distal threads 722 on an external surface thereof having a second pitch and a second incline angle. The thread pitch differential and the opposing or inverted relationship of the thread incline angles of the dual-headed implant 700 may be similar to that of the implant 100 shown in FIGS. 1-5D. The thread pitches may be threaded in substantially the same direction or in opposing directions and may or may not have different pitches. The implant 700 may be constructed of any suitable material such as stainless steel, titanium, or other metals or rigid polymers.

In one embodiment, the distal portion 720 may include a distal driving end 724 having a female depression adaptable to mate with a driver bit 200 (not shown in FIG. 7) having a male interface 210 (not shown in FIG. 7). The proximal portion 710 may include a proximal driving end 714 having a male extension adaptable to mate with a driver bit 200 having a corresponding female depression (not shown in FIG. 7). For example, the distal driving end 724 may define a hex-shaped depression, whereby a suitable driver has a corresponding male hex adapter appropriate to drive the proximal portion 710 of the dual-headed implant 700 into a respective bone. Likewise, the proximal driving end 714 may have a hex-shaped male extension, whereby a suitable driver as a corresponding female hex adapter appropriate to drive the distal portion 720 of the dual-headed implant into a respective bone.

FIGS. 8A and 8B show an exemplary method of inserting a dual-headed implant 700 according to the present subject matter, with reference to the same toe 500 shown in FIGS. 5A-5D. With reference to FIG. 8A, in one embodiment to install a dual headed implant 700, the toe 500 may be surgically opened to provide access to a proximal inter-phalangeal (PIP) joint between a proximal phalanx 510 and a middle phalanx 520. The proximal and middle bone surfaces 512 and 522, respectively, of the proximal 510 and middle 520 phalanges, may be resected using a bone saw or other tool if necessary.

A distal intramedullary canal 524 may be drilled into the tip 502 of the toe 500, through distal phalanx 530 and middle phalanx 520, and out the middle bone surface 522. Alternatively, the drilling spade end 220 of a first drill bit 200 can be used to drill the intramedullary canal 524 into the middle phalanx, through the distal phalanx 530, and out the tip 502 of the toe 500, leaving the male extension 210 proud of the middle bone surface 522. The distal portion 720 of the dual-headed implant 700, having a female depression, is then mated to the male extension 210 of the driver bit 200 and introduced into the middle phalanx 520 through the middle bone surface 522. Where the proximal portion 710 of the dual headed implant 700 has a proximal driving end 714 with a male extension, the driver 300 is adapted with a second driving bit 200 having a corresponding female interface 212. The female interface 212 engages the proximal driving end 714 and drives the distal portion 720 into the middle phalanx 520, such that the distal threads 722 penetrate the bone tissue, until the proximal portion 710 is just proud of the middle bone surface 522. The driver 300 and second driver bit 200 are then removed.

With reference to FIG. 8B, the toe 500 is then aligned in preparation for the proximal phalanx 510 to receive the proximal portion 710 of the dual-headed implant 700, so as to introduce the proximal driving end 514 into the proximal phalanx 520 through the proximal bone surface 522. The driver 300 is then reconfigured to be adapted with the first driver bit 200. The dual-headed implant 700 is then driven towards the proximal phalanx 510 such that the proximal threads 712 penetrate the bone tissue of the proximal phalanx 510. The dual-headed implant 700 is optimally placed when the transition between the proximal portion 710 and distal portion 720 is aligned with the fused PIP joint. The driving bit 200 is then separated from the implant 700 and withdrawn through the tip 502 of the toe 500.

Although the retrograde motion of the dual-headed implant 700 being driven into the proximal phalanx 510 may result in the distal portion 720 partially backing out of the middle phalanx 520, the thread pitch differential between the proximal and distal threads, 712 and 722, ensures that the proximal portion 710 advances more with each turn than the distal portion 720 retreats. The overall motion of the dual-headed implant 700 thus pulls the middle phalanx 520 and proximal phalanx 510 towards each other until the proximal and middle bone surfaces, 512 and 522, are adjacent and the PIP joint is compressed.

In all of the embodiments described, the use of reverse incline thread angles will prevent the implant from pistoning as the toe moves, and will help maintain placement of the implant during walking and other activities by virtue of the compressive force across the PIP joint. The angle of the threads resists movement of the bones against the direction of the compressive force created by the thread pitch differential, thus preventing the implant from moving or the proximal and middle phalanges from separating.

Although reference has been made to a patient's proximal and middle phalanges and PIP joint, one skilled in the art will understand that embodiments of the present subject matter may be implemented for other respective bones including, but not limited to other phalanges/digits and phalangeal/digital joints. Additionally, although reference has been made to the implants of the present subject matter having proximal and distal portions with corresponding proximal and distal structures, these are merely relative terms used for clarity. One skilled in the art will understand that embodiments of the present subject matter encompass any implants having first and second portions, with corresponding first and second structures, as described in the various figures.

It may be emphasized that the above-described embodiments, particularly any “preferred” embodiments, are merely possible examples of implementations and merely set forth for a clear understanding of the principles of the disclosure. Many variations and modifications may be made to the above-described embodiments of the disclosure without departing substantially from the spirit and principles of the disclosure. For instance, one of skill will appreciate that the steps of the various methods described herein may be performed in different orders than the order in which they are described and claimed. All such modifications and variations are intended to be included herein within the scope of this disclosure and the present disclosure and protected by the following claims.

While this description contains many specifics, these should not be construed as limitations on the scope of the claimed subject matter, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

While preferred embodiments of the present subject matter have been described, it is to be understood that the embodiments described are illustrative only and that the scope of the invention is to be defined solely by the appended claims when accorded a full range of equivalence, many variations and modifications naturally occurring to those of skill in the art from a perusal hereof.

We claim:

1. A bone implant comprising:
an elongated body having a first portion bearing a first thread, and a second portion bearing a second thread,
wherein the first thread has a first thread pitch and a first thread angle,
wherein the second thread has a second thread pitch and a second thread angle,
wherein the first thread angle and second thread angle are in an opposing configuration, and
wherein the first portion has a driving end adapted to mate with a driver.

2. The bone implant of claim 1 wherein the first thread pitch is 0.039 inches.

3. The bone implant of claim 1 wherein the second thread pitch is 0.069 inches.

4. The bone implant of claim 1 wherein the first thread angle is 25 degrees from perpendicular to a longitudinal axis of the body.

5. The bone implant of claim 1 wherein the second thread angle is 115 degrees from a longitudinal axis of the body.

6. The bone implant of claim 1 wherein the driving end defines a female depression configured to mate with a male driver bit.

7. The bone implant of claim 1 wherein the body further comprises an unthreaded transition between the first portion and the second portion.

8. The bone implant of claim 1 wherein the first portion is implanted into a patient's proximal phalanx, and the second portion is implanted into the patient's middle phalanx.

9. The bone implant of claim 8 wherein the bone implant creates a compressive force across the patient's proximal inter-phalangeal joint.

10. A bone implant comprising:
an elongated body having a first portion bearing a first thread and defining a first driving end, and a second portion bearing a second thread and defining a second driving end,
wherein the first thread has a first thread pitch and a first thread angle,
wherein the second thread has a second thread pitch and a second thread angle, and
wherein the first driving end is adapted to mate with a first driver bit and the second driving end is adapted to mate with a second driver bit.

11. The bone implant of claim 10 wherein the first thread pitch is 0.039 inches.

12. The bone implant of claim 10 wherein the second thread pitch is 0.069 inches.

13. The bone implant of claim 10 wherein the first thread angle and the second thread angle are in an opposing configuration.

14. The bone implant of claim 10 wherein the first driving end defines a female depression configured to mate with a male driver bit and the second driving end has a male extension configured to mate with a female driver bit.

15. The bone implant of claim 10 wherein the body defines a cannula therethrough.

16. The bone implant of claim 10 wherein the body further comprises an unthreaded transition between the first portion and the second portion.

17. The bone implant of claim 10 wherein the first portion is implanted into a patient's middle phalanx and the second portion is implanted into a patient's proximal phalanx.

18. The bone implant of claim 17 wherein the bone implant creates a compressive force across the patient's proximal inter-phalangeal joint.

19. A method of correcting hammertoes comprising the steps of:
making a dorsal incision in a patient's toe along the patient's proximal inter-phalangeal joint,
bending the patient's toe such that the proximal inter-phalangeal joint is open and a part of the proximal and middle phalanges are exposed,
inserting into the proximal phalanx a bone implant having a first portion bearing a first thread and a second portion bearing a second thread and defining a driving end, such that the first portion penetrates the proximal phalanx and the first thread engages bone tissue of the proximal phalanx,
drilling an intramedullary canal through the patient's distal and middle phalanges,
inserting a driver bit adapted to mate with the driving end into the intramedullary canal through the patient's toe tip,
straightening the patient's toe such that the second portion of the bone implant aligns with the intramedullary canal,
driving the bone implant into the middle phalanx by mating the driver bit with the driving end, until the second portion is anchored in the middle phalanx and the proximal inter-phalangeal joint is compressed, and
withdrawing the driver bit through the patient's toe tip.

20. The method of claim 19 further comprising the step of drilling a second intramedullary canal in the proximal phalanx, before inserting the first portion of the implant into the proximal phalanx.

21. A method of correcting hammertoes comprising the steps of:
making a dorsal incision in a patient's toe along the patient's proximal inter-phalangeal joint,
bending the patient's toe such that the proximal inter-phalangeal joint is open and a part of the proximal and middle phalanges are exposed,
drilling an intramedullary canal through the patient's distal and middle phalanges,
placing in the intramedullary canal a first driver bit adapted to mate with the first driving end,
inserting into the middle phalanx, through the intramedullary canal, a bone implant having a first portion bearing a first thread and defining a first driving end and a second portion bearing a second thread and defining a second driving end, such that the first driving end penetrates the middle phalanx and the first thread engages the bone tissue of the middle phalanx,
mating with the first driving end the first driver bit adapted to mate with the first driving end,
driving the bone implant distally into the middle phalanx by mating with the second driving head a second driver bit adapted to so mate, until the first portion is anchored in the middle phalanx and the second portion is just proud of the proximal inter-phalangeal joint,
removing the second driver bit,
straightening the patient's toe and inserting the second portion of the bone implant into the proximal phalanx such that the second driving end penetrates the proximal phalanx and the second thread engages the bone tissue of the proximal phalanx,
driving the bone implant into the proximal phalanx by rotating in the opposite direction the first driver bit adapted to mate with the first driving end, until the second portion is anchored in the proximal phalanx and the proximal inter-phalangeal joint is compressed, and
withdrawing the first driver bit through the patient's toe tip.

22. The method of claim 21 further comprising the step of drilling a second intramedullary canal in the proximal phalanx, before inserting the second portion of the implant into the proximal phalanx.

23. The implant of claim 1 wherein the body defines a cannula therethrough.
